(19) United States
(12) Patent Application Publication
(10) Pub. No.: US 2014/0147903 A1
(43) Pub. Date: May 29, 2014

(54) Process for the Preparation of Amoebocyte Lysate from Haemolymph of the Horseshoe Crab

(71) Applicants: Ismail Noraznawati, Terengganu (MY); Taib Mariam, Kuala Terengganu (MY); Ahmed Shamsuddin Ahmed, Kuala Terengganu (MY); Chatterji Anil, Goa (IN)

(72) Inventors: Ismail Noraznawati, Terengganu (MY); Taib Mariam, Kuala Terengganu (MY); Ahmed Shamsuddin Ahmed, Kuala Terengganu (MY); Chatterji Anil, Goa (IN)

(21) Appl. No.: 13/882,313

(22) PCT Filed: Oct. 27, 2011
(86) PCT No.: PCT/MY11/00226

§ 371 Date: May 22, 2013

(30) Foreign Application Priority Data
Oct. 29, 2010 (MY) PI2010005112

(51) Int. Cl. C12N 13/00

(52) U.S. Cl. CPC C12N 13/00
USPC 435/173.7

(57) ABSTRACT

The present invention relates to a non-destructive process for the preparation of amoebocyte lysate from the haemolymph of horseshoe crab, characterized by the steps of firstly, means for acclimatizing a live specimen of horseshoe crab in treated seawater under controlled conditions; then, means for cleaning the live specimen, including cleaning the base of the last pair of thoracic appendages of the live specimen; next, means for withdrawing the haemolymph through the base of the last pair of thoracic appendage under aseptic condition; then, means for transferring the collected haemolymph into a pre-cooled disposable centrifuge tube; then, means for centrifuging the collected haemolymph to separate amoebocyte cells from lymph; then, means for decanting the lymph out of the disposable centrifuge tube; then, means for washing the white amoebocyte cells and centrifuging; then, means for sonicating the amoebocyte cells for lysing of the amoebocyte cells; then, means for storing the sonicated amoebocyte cells for further lysing of the amoebocyte cells; then, means for centrifuging the sonicated mixture, thereby separating the amoebocyte lysate in the form of a supernatant; then, means for decanting the amoebocyte lysate into a sterilized vessel; then, means for pre-freezing the amoebocyte lysate; and subsequently, freeze-drying the amoebocyte lysate.

### BACKGROUND OF THE INVENTION

1. Field of the Invention

This invention relates to the preparation of amoebocyte lysate from the haemolymph of horseshoe crab, and more particularly to a non-destructive process for preparation of amoebocyte lysate from the haemolymph of horseshoe crab without using anti-coagulant agent.

2. Description of Related Arts

Gram-negative bacteria naturally occur in the air we breathe and in the water we drink daily. They are even found in our intestine tract. People have a mechanism regulated by the liver that prevents absorption of the bacteria from the gastrointestinal tract into the blood system, so under normal condition or healthy circumstances, Gram-negative bacteria pose no threat to people. However, if the bacteria have an opportunity to enter the blood stream, such as in the case of trauma, they are highly potential to cause fatal fevers. Gram-negative diseases include toxic-shock syndrome, spinal meningitis, typhoid, and gonorrhea. Scientists discovered that the fevers were caused by endotoxins which are found in the cell walls of Gram-negative bacteria.

As is well known, amoebocyte lysate is useful as a testing reagent for the detection of Gram-negative bacterial contamination. In addition, the said reagent is highly sensitive and useful for the rapid and accurate assay of Gram-negative bacteria even if they are present in a very minute quantity up to the level of 10⁻¹⁰ g. Moreover, the amoebocyte lysate has been proved to be a valuable diagnostic reagent in the detection of endotoxins in several pharmaceutical products especially injectable vaccines. The diagnosis of Gram-negative bacteria disease is very quick and effective by this reagent as compared to the normal Rabbit Pyrogen Test.

However, process for the preparation of amoebocyte lysate from the haemolymph of the horseshoe crab are known in the prior art. For example, U.S. Pat. No. 3,954,663 disclosed a process for the preparation of lysate which is useful for the detection of endotoxin. The process comprising extracting the blood of horseshoe crab (Limulina) into an isotonic buffer solution containing methyl derivative of xanthine as amoebocyte's agglutination-inhibiting agent, separating the amoebocyte from said solution, breaking the amoebocyte, and recovering Limulina lysate. However, it is preferable to avoid using the anti-coagulant agent during the preparation of amoebocyte lysate which may pose the chances of contamination of amoebocyte lysate. Furthermore, the said agent may also complicate the process and is cost consuming.

In other example, U.S. Pat. No. 5,401,647 disclosed a method for preparing limulus amoebocyte lysate substantially free from factor G which comprises bringing limulus amoebocyte lysate into contact with an insoluble carrier. The amoebocyte lysate is first activated with (1±3)-β-D-glucan in the limulus amoebocyte lysate coagulation mechanism for removing factor G. However, the drawback of this patent includes several steps of removal of factor G making the process more cumbersome.

The process of preparation of amoebocyte lysate was developed for the first time by Jorgenson and Smith (1973). Jorgenson, J. H. et al, disclosed the Limulus assay has been used as a method for detecting endotoxin in patients with gram-negative septicaemia and as a method for detecting pyrogen in parenteral pharmaceuticals. The haemolymph was allowed to flow directly into pyrogen-free siliconized polypropylene centrifuge bottles which containing N-ethylmaleimide to prevent amoebocytes agglutination. However, this process leads to the disadvantage of the removal of haemolymph resulted in the death of the horseshoe crab.

Therefore, there is a need of present invention to provide a non-destructive extraction of amoebocyte lysate from the haemolymph of the horseshoe crab without using any anti-coagulant agent to make the process easier and cheap. Moreover, this process also helps to conserve the valuable biodiversity in worldwide.

### REFERENCES

Jorgenson, J. H. et al. 1973. Preparation, Sensitivity, and Specificity of Limulus Lysate for Endotoxin Assay. American Society for Microbiology, Volume 26. Page 43-48.

### SUMMARY OF INVENTION

It is an object of the present invention to provide a non-destructive process for the preparation of amoebocyte lysate from the haemolymph of horseshoe crab without using amoebocyte's agglutination-inhibiting agent so as to considerably reduce the production cost and chances of contamination.

It is also an object of the present invention to provide a non-destructive process for the preparation of amoebocyte lysate from the haemolymph of horseshoe crab without sacrificing the animals to conserve the valuable biodiversity.

It is yet another object of the present invention to provide a non-destructive process for the preparation of amoebocyte lysate from the haemolymph of horseshoe crab which having high sensitivity.

It is a further object of the present invention to provide a non-destructive process for the preparation of amoebocyte lysate from the haemolymph of horseshoe crab that can avoid some cumbersome processes such as removal of factor G.

Accordingly, these objectives may be achieved by following the teachings of the present invention. The present invention relates to a process for producing amoebocyte lysate from haemolymph of horseshoe crab. The amoebocyte lysate is highly sensitive and useful for the rapid and accurate assay of Gram-negative bacteria even if they are present in a very minute quantity up to the level of 10⁻¹⁰ g. The present process comprises collection of the amoebocyte lysate from live specimens of the horseshoe crab under highly aseptic condition without the usage of anti-coagulants. Firstly, acclimatizing a live specimen of horseshoe crab in treated seawater under controlled conditions; then, cleaning the live specimen, including cleaning the base of the last pair of thoracic appendages of the live specimen; next, withdrawing the haemolymph from the live specimen through the base of the last pair of thoracic appendage under aseptic condition; then, transferring the collected haemolymph into a pre-cooled disposable centrifuge tube under aseptic condition; then, centrifuging the collected haemolymph to separate amoebocyte cells from lymph; then, decanting the lymph out of the disposable centrifuge tube; then, washing the white amoebocyte cells by using sterilized double-distilled water and centrifuging; then, sonicating the amoebocyte cells with sterilized double-distilled water for lysing of the amoebocyte cells; then, storing the sonicated amoebocyte cells for further lysing of the amoebocyte cells; then, centrifuging the sonicated mixture, thereby separating the amoebocyte lysate in the form of a supernatant; then, decanting the amoebocyte lysate into a sterilized vessel; then, pre-freezing the amoebocyte lysate; and subsequently, freeze-drying the amoebocyte lysate.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting but merely as a basis for claims. It should be understood that the drawings and detailed description thereto are not intended to limit the invention to the particular form disclosed, but on the contrary, the invention is to cover all modification, equivalents and alternatives falling within the spirit and scope of the present invention as defined by the appended claims. As used throughout this application, the word “may” is used in a permissive sense (i.e., meaning having the potential to), rather than the mandatory sense (i.e., meaning must). Similarly, the words “include,” “including,” and “includes” mean including, but not limited to. Further, the words “a” or “an” mean “at least one” and the word “plurality” means one or more, unless otherwise mentioned. Where the abbreviations of technical terms are used, these indicate the commonly accepted meanings as known in the technical field.

The present invention relates to a process for producing amoebocyte lysate from haemolymph of horseshoe crab. The amoebocyte lysate is highly sensitive and useful for the rapid and accurate assay of Gram-negative bacteria even if they are present in a very minute quantity up to the level of 10⁻¹⁰ g. The present process comprises collection of the amoebocyte lysate from live specimens of the horseshoe crab under highly aseptic condition without the usage of anti-coagulants. Firstly, means for acclimatizing a live specimen of horseshoe crab in treated seawater under controlled conditions; then, means for cleaning the live specimen, including cleaning the base of the last pair of thoracic appendages of the live specimen; next, means for withdrawing the haemolymph from the live specimen through the base of the last pair of thoracic appendage under aseptic condition; then, means for transferring the collected haemolymph into a pre-cooled disposable centrifuge tube under aseptic condition; then, means for centrifuging the collected haemolymph to separate amoebocyte cells from lymph; then, means for decanting the lymph out of the disposable centrifuge tube; then, means for washing the white amoebocyte cells by using sterilized double-distilled water and centrifuging; then, means for sonicating the amoebocyte cells with sterilized double-distilled water for lysing of the amoebocyte cells; then, means for storing the sonicated amoebocyte cells for further lysing of the amoebocyte cells; then, means for centrifuging the sonicated mixture, thereby separating the amoebocyte lysate in the form of a supernatant; then, means for decanting the amoebocyte lysate into a sterilized vessel; then, means for pre-freezing the amoebocyte lysate; and freeze-drying the amoebocyte lysate.

Below is an example of a non-destructive process for the preparation of amoebocyte lysate from the haemolymph of horseshoe crab from which the advantages of the present invention may be more readily understood. It is to be understood that the following example is for illustrative purpose only and should not be construed to limit the present invention in any way.

### EXAMPLES

The collection of live horseshoe crab specimens are done at the sandy beach of Penisular Malaysia, wherein the horseshoe crabs are Tachypleus gigas Muller, Caminoscorpuis rotundicauda Latreilli and Tachypleus tridentatus Leach. The healthy specimens are maintained under controlled conditions in seawater. The animals were allowed to acclimate for 48 hours at ambient temperature before they were used for the collection of haemolymph.

The Malaysian horseshoe crabs at first were sterilized by wiping with a piece of cotton sobbed in 70% alcohol. The base of the last pair of thoracic appendages was cleaned thoroughly with alcohol for two to three times. Haemolymph, approximately 20 ml, withdrawn through the base of the last pair of thoracic appendage with a sterilised 16 gauge stainless steel needle fitted to a disposable hypodermic syringe into a pre-cooled disposable centrifuge tube under aseptic condition. Meanwhile, the bled animals were then immediately transferred to culture tank for safe recovery.

The collection of haemolymph was centrifuged at 4° C. at a constant speed of 72×g for 15 minutes using a refrigerated centrifuge. The supernatant containing the lymph was slowly decanted in another capped sterilized glass bottle and kept separately for further use. The separated pellet containing white amoebocyte cells were washed for two times by sterilized double distilled water and then centrifuged for at least 15 minutes at a speed of 290×g and at temperature 4° C. Next, the amoebocyte cells were sonicated in the presence of 2 ml sterilized double distilled water and kept at 4° C. in a refrigerator for 10 hours for the maximum lysing of amoebocyte cells. The sonicated mixture was then centrifuged again at 4° C. for at least 15 minutes at a constant speed of 1157×g, thereby separating the amoebocyte lysate in the form of supernatant. The supernatant was then decanted slowly into a sterilized vial fitted with a slotted rubber cap and termed as amoebocyte lysate. The amoebocyte lysate was kept for at least 24 hours at −80° C. before being freeze dried purpose by using a table top freeze dryer under vacuum condition. The said freeze dried amoebocyte lysate can be resuspended in sterilized double-distilled water.

The amoebocyte lysate so prepared was used for carrying out the sensitivity assay of amoebocyte lysate against endotoxins. The lyophilized sample of amoebocyte lysate was resuspended in 1 ml sterilized double distilled water, whereas the lyophilized samples of endotoxins, preferably E. coli, Salmonella sp., Pseudomonas sp. and Klebseilla sp. were obtained from the market and resuspended in sterilized double distilled water as per instructions of the manufacturers. Both reconstituted material were thoroughly vortexed to get the materials fully dissolved. These stock solutions of each bacterial strain, serial dilution (10⁻¹, 10⁻², 10⁻³, 10⁻⁴, and 10 endotoxin samples were prepared in double distilled water and kept at temperature 4° C. for further use.

Each concentration of endotoxin of different strains (0.1 ml) and amoebocyte lysate (0.1 ml) were transferred to a glass endotoxin tube and both solutions were vortexed thoroughly. In one other endotoxin test tube only 0.1 ml endotoxin and 0.1 ml sterilized distilled water was kept and treated as negative control. Meanwhile, 0.1 ml of different concentrations of endotoxin with commercial Limulus amoebocyte lysate obtained from the market to act as positive control. AH these endotoxin glass tubes were kept at 37 for 2 hours for incubation. AH the incubated endotoxin test tubes were then examined for the rate of agglutination reaction as described by Jorgenson and Smith (1973) to access the sensitivity of the amoebocyte lysate. Based on the results from this investigation, it is shown that for all endotoxin concentrations of different bacterial strains, the sensitivity of amoebocyte lysate was very high compared to the sensitivity of the commercial Limulus amoebocyte lysate.

I/we claim:

1. A non-destructive process for the preparation of amoebocyte lysate from the haemolymph of horseshoe crab, characterized by the steps of:
firstly, means for acclimatizing a live specimen of horseshoe crab in treated seawater under controlled conditions;
then, means for cleaning the live specimen, including cleaning the base of the last pair of thoracic appendages of the live specimen;
then, means for withdrawing the haemolymph from the live specimen through the base of the last pair of thoracic appendage under aseptic condition;
then, means for transferring the collected haemolymph into a pre-cooled disposable centrifuge tube under aseptic condition;
then, means for centrifuging the collected haemolymph to separate amoebocyte cells from lymph;
then, means for decanting the lymph out of the disposable centrifuge tube;
then, means for washing the white amoebocyte cells by using sterilized double-distilled water and centrifuging;
then, means for sonicating the amoebocyte cells with sterilized double-distilled water for lysing of the amoebocyte cells;
then, means for storing the sonicated amoebocyte cells for further lysing of the amoebocyte cells;
then, means for centrifuging the sonicated mixture, thereby separating the amoebocyte lysate in the form of a supernatant;
then, means for decanting the amoebocyte lysate into a sterilized vessel;
subsequently, means for pre-freezing the amoebocyte lysate; and freeze-drying the amoebocyte lysate.

2. A non-destructive process for the preparation of amoebocyte lysate from the haemolymph of horseshoe crab according to claim 1, wherein the said horseshoe crab is preferably Tachypleus gigas, Cartinoscorpuis rotundicauda and Tachypleus tridentatus.

3. A non-destructive process for the preparation of amoebocyte lysate from the haemolymph of horseshoe crab according to claim 1, wherein means for cleaning the live specimen is carried out preferably with sterilized double-distilled water and alcohol.

4. A non-destructive process for the preparation of amoebocyte lysate from the haemolymph of horseshoe crab according to claim 1, wherein the live specimen is preferably acclimatized in treated seawater for at least 48 hours and at ambient temperature.

5. A non-destructive process for the preparation of amoebocyte lysate from the haemolymph of horseshoe crab according to claim 1, wherein the withdrawal of haemolymph from the live specimen is preferably carried out by using a sterilised 16 gauge stainless steel needle fitted to a disposable hypodermic syringe.

6. A non-destructive process for the preparation of amoebocyte lysate from the haemolymph of horseshoe crab according to claim 1, wherein the collected haemolymph is preferably centrifuged at 72×g and 4° C. for at least 15 minutes.

7. A non-destructive process for the preparation of amoebocyte lysate from the haemolymph of horseshoe crab according to claim 1, wherein the means for washing of the amoebocyte cells is carried out preferably but not limited for two times by using sterilized double-distilled water and centrifuging at 290×g and 4° C. for at least 15 minutes.

8. A non-destructive process for the preparation of amoebocyte lysate from the haemolymph of horseshoe crab according to claim 1, wherein the sonication of the amoebocyte cells is preferably carried out with 2 ml of sterilized double-distilled water.

9. A non-destructive process for the preparation of amoebocyte lysate from the haemolymph of horseshoe crab according to claim 1, wherein the sonicated amoebocyte cells are preferably stored at 4° C. for at least 10 hours for further lysing of the amoebocyte cells.

10. A non-destructive process for the preparation of amoebocyte lysate from the haemolymph of horseshoe crab according to claim 1, wherein the sonicated mixture is preferably centrifuged at 1157×g and 4° C. for at least 15 minutes.

11. A non-destructive process for the preparation of amoebocyte lysate from the haemolymph of horseshoe crab according to claim 1, wherein the means for pre-freezing of the amoebocyte lysate is preferably carried out at −80° C. for at least 24 hours.

12. A non-destructive process for the preparation of amoebocyte lysate from the haemolymph of horseshoe crab according to claim 1, wherein the preparation of amoebocyte lysate may be further freeze-dried under vacuum condition.
